(19) United States
(12) Patent Application Publication
(10) Pub. No.: US 2010/0189694 A1
(43) Pub. Date: Jul. 29, 2010

(54) Scar Tissue Treatment System

(75) Inventor: Mark A Palmer, Dewsbury (GB)

Correspondence Address: CR MILES, P.C.;CRAIG R. MILES, 405 MASON COURT, SUITE 119, FORT COLLINS, CO, 80524, US

(73) Assignee: Alizonne (UK) Ltd.

(21) Appl. No.: 12/226,329

(22) PCT Filed: Oct. 17, 2007
(86) PCT No.: PCT/IB2007/004544

§ 371 Date: Oct. 15, 2008

(51) Int. Cl. A61K 35/36

(52) U.S. Cl. 424/93.7; 435/325

(57) ABSTRACT

A treatment for hypertrophic scar tissue and methods for treatment of hypertrophic scar tissues utilizing viable fibroblast cells.

### I. TECHNICAL FIELD

Generally, a method for treatment of hypertrophic scar tissue utilizing viable fibroblast cells.

### II. BACKGROUND

Fibroblast cells are naturally present in the dermis, or inner layer of the skin as well other tissues such as the buccal mucosa of the mouth and are responsible for the production of collagen and elastin fibers which are the main structural components of the dermis. In addition, fibroblasts cells are essential to and active in the wound healing process involving the production of new collagen and elastin fibers following trauma.

Abnormal scarring can result from the localized overproduction of collagen or elastin fibers during the healing process which can cause the tissue to be thickened, raised or elevated above the surrounding skin. Hypertrophic scars typically take the form of a thickened area or raised lump on the skin which does not grow beyond the boundaries of the original wound. The tissue structure of hypertrophic scars can be inelastic and may contract or shorten over time causing traction or tension on the surrounding tissues which can cause pain, restriction of movement and a resultant loss of function in the affected parts of the body. Conventional treatment of hypertrophic scars includes removal of the abnormal tissue structure and reconstruction with skin grafts.

Treatment of hypertrophic scars has not included the use of fibroblast cells. The Conventional wisdom teaches away from treatment of hypertrophic scars because treatment of tissue with fibroblast cells results in production of additional collagen and elastin fibers. It is conventionally believed that treatment of hypertrophic scars with fibroblast cells will result in additional production of collagen and elastin fibers in areas at which there is already an abnormal excess of collagen and elastin fibers. Therefore, it is conventionally thought that treatment of hypertrophic scars with fibroblast cells will increase the severity of hypertrophic scarring. Accordingly, conventional use of fibroblast cells for treatment of scar tissue has been restricted to treatment of atrophic scars which represent localized areas of inadequate healing with underproduction of collagen and elastin fibers which can result in tissue which is thinner than surrounding unscarred dermis and which can appear as poorly healed, thinned, or depressed areas, valleys, pockets or holes.

The present inventive method which teaches away from conventional wisdom provides a scar tissue treatment system for the treatment of hypertrophic scar tissue with fibroblast cells.

### III. DISCLOSURE OF INVENTION

A broad object of the invention can be to provide a method for treatment of hypertrophic scar tissue in animals utilizing viable fibroblast cells. The term “fibroblast cell” means a cell present in connective tissue capable of forming collagen fibers and without limitation includes autologous fibroblast cells obtained from the animal to be treated by the inventive method or allogenic fibroblast cells obtained from another one of the same species, or a different species of animal. The term “viable” means retention of fibroblast cell functions which allow the production collagen fibers. The term “animal” means any manner of animal capable of producing fibroblast cells, including, but not limited to, humans, cattle, horses, dogs, and cats. The term “hypertrophic scar” or “hypertrophic scar tissue” means any tissue that is thickened due to localized overproduction of collagen and elastin fibers, and in part includes hypertrophic restrictive scar tissue which can be structurally inelastic to an extent which may result in traction or tension on surrounding tissues which can cause pain or restrict movement of the animal.

Another broad object of the invention can be to provide an efficacious concentration of fibroblast cells whether autologous or allogenic for the treatment of hypertrophic scar tissue and hypertrophic restrictive scar tissue.

Naturally, further objects of the invention are disclosed throughout other areas of the specification and drawings.

### IV. BRIEF DESCRIPTION OF THE DRAWINGS

FIG. 1 is a block diagram of the steps of a method for treatment of hypertrophic scar tissue.

FIG. 2 is a diagram showing the method of delivering a dose to hypertrophic scar tissue.

### V. MODE(S) FOR CARRYING OUT THE INVENTION

A treatment for hypertrophic scar tissue and methods for treatment of hypertrophic scar tissues utilizing viable fibroblast cells.

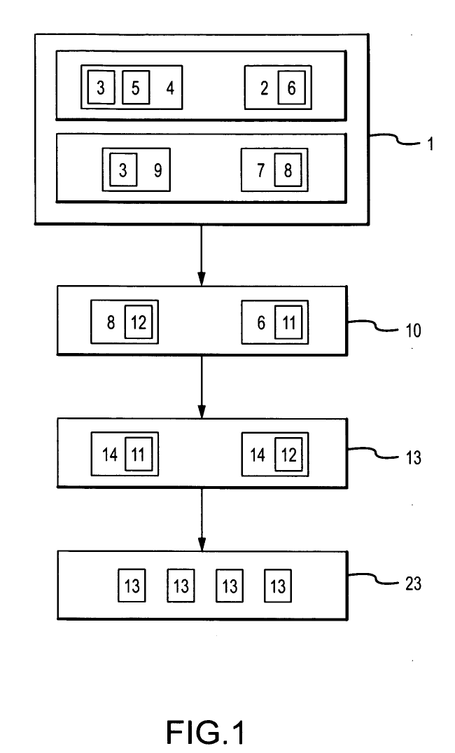

Now referring primarily to FIG. 1, a block diagram sets out the steps of a method for treatment of hypertrophic scar tissue (5). In a first method step (1) of generating a plurality of viable fibroblast cells, an autologous animal fibroblast cell culture (2) can be initiated by obtaining a biopsy material (3) from a hypertrophic scarred animal (4) having an amount of the hypertrophic scar tissue (5) to produce a plurality of autologous animal fibroblast cells (6). Alternately, embodiments of the inventive method can also include as to the first method step (1) an allogenic animal fibroblast cell culture (7) initiated by obtaining a biopsy material (3) from a donor animal (9) separate from the hypertrophic scarred animal (4) having the hypertrophic scar tissue (5) to produce a plurality of allogenic animal fibroblast cells (8).

The biopsy material (3) can be obtained from various locations on the hypertrophic scarred animal (4) or donor animal (9). Typically, the biopsy material (3) is a skin biopsy material, although the invention is not so limited, and the biopsy material (3) can be any tissue or material which produces or is capable of transferring fibroblast cells of the hypertrophic scarred animal (4) or the donor animal (9) as a source of autologous or allogenic fibroblast cells to initiate the autologous animal fibroblast cell culture (2) or the allogenic animal fibroblast cell culture (7). As non-limiting examples, in cattle embodiments of the inventive method, the biopsy material (3) may come from the ear of a bovid, while in human embodiments of the inventive method, the biopsy material (3) can be obtained from the buccal mucosa inside the lip or cheek or can be obtained from the skin of the forehead, or the skin in the crease immediately behind the earlobe (pinna) of a human.

Various methods for isolation of fibroblast cells from the skin biopsy material (3) are known by those of skill in the fibroblast cell culture art. One non-limiting example of isolating fibroblast cells for culture can be to scissor-mince the biopsy material (3) into smaller pieces and to allow these smaller pieces of biopsy material (3) to adhere to the base of a conventionally configured tissue culture flask, such as a T-flask. When the smaller pieces of the biopsy material (3) have adhered to the culture flask, the cells resident in the biopsy material (3) (keratinocyte cells from the epidermis and fibroblast cells from the dermis) can migrate out of the skin material (3). More complex methods of obtaining fibroblast cells involve removal of the epidermal keratinocyte cell layer by enzymatic treatment using dispase, trypsin, collagenase, or the like, followed again by explant culture and differential cell growth culture conditions.

The autologous animal fibroblast cell culture (2) or allogenic animal fibroblast cell culture (7) conditions can be altered by changing the constituents of the liquid media in which cells are grown or by the length of time the cells are grown, or both in various permutations and combinations, to encourage growth of one or the other of the cell types (keratinocytes from the epidermis or fibroblasts from the dermis). Once cell culture conditions are established fibroblast cells can proliferate to provide the plurality of autologous fibroblast cells (6) or the plurality of allogenic fibroblast cells (8) utilized in the inventive methods of treatment for hypertrophic scar tissue described herein. The plurality of autologous fibroblast cells (6) or the plurality of allogenic fibroblast cells (8) cultured can be harvested and utilized directly according to the methods below described or stored in liquid nitrogen until used.

The inventive method for the treatment of hypertrophic scar tissue (5) can be achieved utilizing the plurality of autologous fibroblast cells (6) or the plurality of allogenic fibroblast cells (8) produced respectively by autologous animal fibroblast cell culture (2) or produced by an allogenic animal fibroblast cell culture (7). The plurality of allogenic fibroblast cells (8) can be as therapeutically efficacious as the plurality of autologous fibroblast cells (6) presumably because the lifespan of the plurality of allogenic fibroblast cells (8) in the hypertrophic scarred animal (4) having hypertrophic scar tissue (5) is sufficiently long to exert efficacious treatment effects through a combination of their own activity before apoptosis or other mode of cell death and through activation and up regulation of native fibroblast cell activity in the hypertrophic scarred animal (4) which can persist for several months. In addition, the plurality of allogenic fibroblast cells (8) may be unrecognized by the immune system of the hypertrophic scarred animal (4).

In a second method step (10) of establishing an efficacious concentration of a plurality of viable fibroblast cells in a diluent, the cultured viable fibroblast cells whether a plurality of autologous fibroblast cells (6) or a plurality of allogenic fibroblast cells (8) can be established at an efficacious concentration of autologous fibroblast cells (11) or can be established at an efficacious concentration of allogenic fibroblast cells (12). The term “diluent” as used herein can include any liquid(s) which can be combined with fibroblast cells or combined with other liquids in which fibroblast cells are suspended to adjust the number of fibroblast cells per milliliter compatible with delivery into hypertrophic scar tissue (5) of a hypertrophic scarred animal (4). As but one non-limiting example, a diluent suitable for use with the invention can be the culture medium used to culture the plurality of autologous fibroblast cells (6) or a plurality of allogenic fibroblast cells (8). One such culture medium which can be used as a diluent can be Dulbecco's Modified Eagles's Medium F-12 available from Invitrogen, 1600 Faraday Avenue, Carlsbad, Calif. As a second example, HYPOTHERMOSOL FRS a preservation solution for cells, tissues, and organs could also be used as the diluent. These two examples are not intended to be limiting and numerous and varied compositions that have similar formulation can be suitable as the diluent depending upon the application.

Typically, an efficacious concentration of autologous fibroblast cells (11) or an efficacious concentration of allogenic fibroblast cells (12) provides at least one million viable autologous fibroblast cells or viable allogenic fibroblast cells per milliliter (“mL”) of diluent with a preferred embodiment of the inventive method providing at least about five million viable autologous fibroblast cells or viable allogenic fibroblast cells per mL of diluent. Certain embodiments of the invention can provide an efficacious concentration of between about one million viable autologous fibroblast cells or viable allogenic fibroblast cells per mL diluent and about forty million viable autologous fibroblast cells or viable allogenic fibroblast cells per mL of diluent. Additional embodiments of the invention can provide a narrower range of between about five million viable autologous fibroblast cells or viable allogenic fibroblast cells per mL of diluent and about twenty million viable autologous fibroblast cells or viable allogenic fibroblast cells per mL of diluent. These examples of an efficacious concentration of viable autologous fibroblast cells (11) or efficacious concentration of viable allogenic fibroblast cells (12) are not meant to limit the efficacious concentration of the plurality of viable fibroblast cells (whether autologous, allogenic, or a combination of autologous and allogenic fibroblast cells) to a particular range, a particular concentration or a particular number, but rather the examples are provided to allow the person of ordinary skill in the art to make efficacious concentrations of fibroblasts cells useful in the treatment hypertrophic scar tissue (5) in a wide variety of applications which may be of greater or lesser number per mL of diluent.

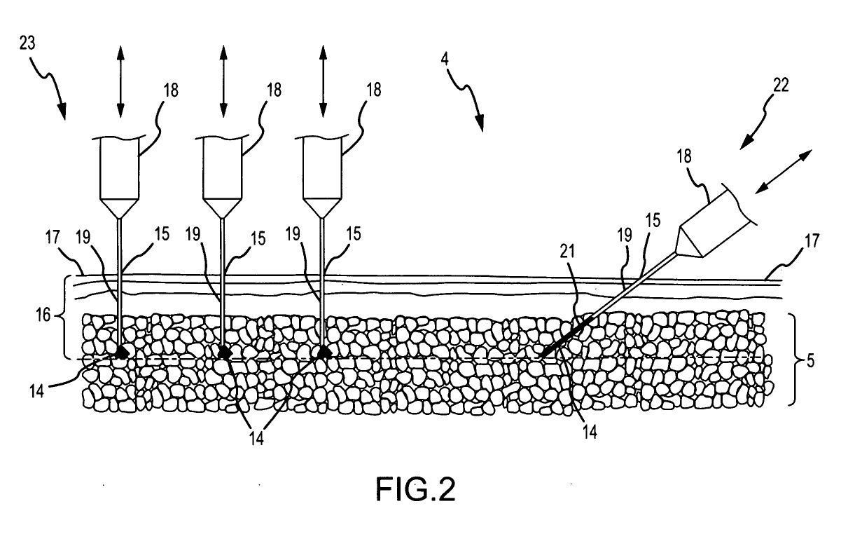

Now referring primarily to FIGS. 1 and 2, a third method step (13) comprises injecting a dose (14) of an efficacious concentration of viable autologous fibroblast cells (11) or an efficacious concentration of viable allogenic fibroblast cells (12) (or a combination of the efficacious concentrations of autologous and allogenic cells) in the hypertrophic scar tissue (5) underlying an injection location (15) on the skin (17) of an animal (4) at an injection location (15) (or at each of a plurality of injection locations from about zero centimeters to about two centimeters (“cm”) apart) at an injection level (16) in the underlying hypertrophic scar tissue (5).

A dose (14) can as a non-limiting example provide an amount the efficacious concentration of viable autologous fibroblast cells (11) or an efficacious concentration of viable allogenic fibroblast cells (12) (or a combination of the efficacious concentrations of autologous and allogenic cells) of between about 0.05 mL and about 0.2 mL. The term “underlying” encompasses that part of the hypertrophic scar tissue (5) located beneath a tissue surface (17) (also referred to as the skin; however, it is to be understood that the term skin encompasses those cases in which the animal is burned to the extent that the skin is replaced entirely or substantially with hypertrophic scar tissue (5)) injectable from a particular injection location (15) regardless of the type of injection needle or the method of injection utilized. The term “an injection level” means the depth at which the dose (14) of an efficacious concentration of viable autologous fibroblast cells (11) or an efficacious concentration of viable allogenic fibroblast cells (12) (or a combination of the efficacious concentrations of autologous and allogenic cells is established in the hypertrophic scar tissue (5).

Now referring primarily to FIG. 2, as but one non-limiting example in humans, the dose (14) of an efficacious concentration of viable autologous fibroblast cells (11) or an efficacious concentration of viable allogenic fibroblast cells (12) (or a combination of the efficacious concentrations of autologous and allogenic cells) can be established into a dose delivery device (18) capable of establishing the dose (14) at an injection level (16) in the hypertrophic scar tissue (5). The dose delivery device (18) can be for example a syringe coupled to an injection needle. The dose (14) can be aspirated into the syringe and injected into the hypertrophic scar tissue (5). Understandably, depending on the volume of the dose (14) the dose delivery device (18) can be of correspondingly lesser or greater volume. As a non-limiting example, a syringe of between one-half mL and a two mL can be fitted with a 13 mm-20 to 32 gauge needle (or other lengths and gauges depending on the application) can be utilized to inject the dose (14) into the hypertrophic scar tissue (5).

Again referring primarily to FIG. 2, the injection location (15) on the tissue surface (17) punctured to inject the dose (14) into the hypertrophic scar tissue (5) underlying the tissue surface (17) can be predetermined with lesser distance between each of a plurality of injection locations (15) in areas of greater hypertrophic scarring and with greater distance apart between each of a plurality of injection locations (15) in areas of lesser hypertrophic scarring, but in any event not greater than two cm apart to avoid areas of untreated hypertrophic scar tissue (5) between injection locations (15) due to a failure or unequal dispersion of the plurality of autologous animal fibroblast cells (6) or the plurality of allogenic animal fibroblast cells (8) contained in the dose (14) through the hypertrophic scar tissue (5) which may result in localized uneven treatment of the tissue about the injection locations (15).

The dose (14) can be delivered at an injection level (16) in the hypertrophic scar tissue (5) by various methods. One method of delivering the dose (14) at an injection level (16) in the hypertrophic scar tissue (5) can be by insertion of a syringe needle (at an angle of about 0 degrees to about 90 degrees to the tissue surface (17)) at the injection level (16) in the hypertrophic scar tissue (5) underlying the injection location (15) which allows substantially the entire dose (14) of between about 0.05 mL and about 0.2 mL to be established in the hypertrophic scar tissue (5) along the withdrawal path (21) of the injection needle (19) of the syringe (20) as it is withdrawn from the hypertrophic scar tissue (5) underlying the injection location (15) (the “needle withdrawal dose delivery method” (22)).

Alternately, delivering a part of the dose (14) at an injection level (16) in the hypertrophic scar tissue (5) underlying a plurality of injection locations (15) (the injection at about 90 degrees to the tissue surface (17)) which encompass a similar sized area of the hypertrophic scar tissue (5) as above treated by the needle withdrawal dose delivery method can also be efficacious in treatment of hypertrophic scar tissue (5). Depending upon the area of hypertrophic scar tissue (5) treated, a greater or lesser number of injection locations (5) can be utilized with a correspondingly greater or lesser dose (14) (or number of doses) injected into the hypertrophic scar tissue (5) underlying the injection locations (5) (“serial puncture method” (23)).

Typically, after elapse of about one week to two weeks following treatment in accordance with the third method step (13) (depending upon the application the duration an vary and in certain application can be greater than two weeks), results may be observed as a softening and flattening of the hypertrophic scar tissue (5) treated along with a reduction in pain associated with a greater range of movement of the tissue responsive to the area of hypertrophic scar tissue (5) treated. Significant flattening of the hypertrophic scar tissue can continue for several months subsequent to the initial treatment in accordance with the third method step (13).

In a fourth method step (23), if desired or necessary, the third method step (13) can be repeated for the same area of hypertrophic scar tissue (5). Typically, elapse of a duration of time of about two weeks to about six weeks between treatments; however, the fourth method step (21) can be performed after the elapse of a greater or lesser amount of time. As one none-limiting example, the third method step (13) of the inventive method of treatment for hypertrophic scar tissue (5) can be repeated four or five times with elapse of about six weeks between repeated application of the third method step (13) for an area having underlying hypertrophic scar tissue (5).

As can be easily understood from the foregoing, the basic concepts of the present invention may be embodied in a variety of ways. The invention involves embodiments of scar tissue treatment system which provides both a fibroblast cell treatment for hypertrophic scar tissue and a method for treatment of hypertrophic scar tissue.

As such, the particular embodiments or elements of the invention disclosed by the description or shown in the figures or tables accompanying this application are not intended to be limiting, but rather exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of a “treatment” should be understood to encompass disclosure of the act of “treating”—whether explicitly discussed or not—and, conversely, were there efficaciously disclosure of the act of “treating”, such a disclosure should be understood to encompass disclosure of a “treatment” and even a “means for treating.” Such alternative terms for each element or step are to be understood to be explicitly included in the description.

In addition, as to each term used it should be understood that unless its utilization in this application is inconsistent with such interpretation, common dictionary definitions should be understood to included in the description for each term as contained in the Random House Webster's Unabridged Dictionary, second edition, each definition hereby incorporated by reference.

Thus, the applicant(s) should be understood to claim at least: i) each of the scar tissue treatments herein disclosed and described, ii) the related methods disclosed and described, iii) similar, equivalent, and even implicit variations of each of these devices and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The background section of this patent application provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent application or continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of the application including any subsequent continuation, division, or continuation-in-part application thereof or any reissue or extension thereon.

Additionally, the claims set forth below are intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

1. A method for treatment of a hypertrophic scar tissue, comprising the steps of:
a) generating a plurality of viable fibroblast cells;
b) establishing an efficacious concentration of said plurality of viable fibroblast cells in an amount of diluent;
c) injecting said hypertrophic scar tissue underlying at least one injection location on the skin of an animal with an amount of said efficacious concentration of said plurality of viable fibroblast cells in said amount of diluent.

2. The method for treatment of a hypertrophic scar tissue as described in claim 1, wherein said step of generating a plurality of viable fibroblast cells comprises the step of culturing a plurality of viable fibroblast cells in an amount of medium.

3. The method for treatment of a hypertrophic scar tissue as described in claim 2, wherein said step of culturing a plurality of viable fibroblast cells in an amount of medium comprises culturing a plurality of viable autologous fibroblast cells in an amount of medium.

4. The method for treatment of a hypertrophic scar tissue as described in claim 2, wherein said step of culturing a plurality of viable fibroblast cells in an amount of medium comprises culturing a plurality of viable allogenic fibroblast cells in an amount of medium.

5. The method for treatment of a hypertrophic scar tissue as described in claim 1, wherein said step of establishing an efficacious concentration of said plurality of viable fibroblast cells in an amount of diluent comprises the step of establishing a concentration of said plurality of viable fibroblast cells in said amount of diluent of not less than about one million viable fibroblast cells per milliliter of said amount of diluent.

6. The method for treatment of a hypertrophic scar tissue as described in claim 1, wherein said step of establishing an efficacious concentration of said plurality of viable fibroblast cells in an amount of diluent comprises the step of establishing a concentration of said plurality of viable fibroblast cells in said amount of diluent of between about one million fibroblast cells per milliliter and about forty million fibroblast cells per milliliter of diluent.

7. The method for treatment of a hypertrophic scar tissue as described in claim 1, wherein said step of establishing an efficacious concentration of said plurality of viable fibroblast cells in an amount of diluent comprises the step of establishing a concentration of said plurality of viable fibroblast cells in said amount of diluent of between about five million fibroblast cells per milliliter and about twenty million fibroblast cells per milliliter of diluent.

8. The method for treatment of a hypertrophic scar tissue as described in claim 1, wherein said step of injecting said hypertrophic scar tissue underlying at least one injection location on the skin of an animal with an amount of said efficacious concentration of said plurality of viable fibroblast cells in said amount of diluent comprises the step of injecting said hypertrophic scar tissue underlying at a plurality of injection locations a distance apart on the skin of an animal with an amount of said efficacious concentration of said plurality of viable fibroblast cells in said amount of diluent.

9. The method for treatment of a hypertrophic scar tissue as described in claim 8, wherein said step of injecting said hypertrophic scar tissue underlying a plurality of injection locations a distance apart on the skin of an animal with an amount of said efficacious concentration of said plurality of viable fibroblast cells in said amount of diluent comprises injecting said hypertrophic scar tissue underlying a plurality of injection locations between about zero centimeter and about two centimeters distance apart on the skin of an animal with an amount of said efficacious concentration of said plurality of viable fibroblast cells in said amount of diluent.

10. The method for treatment of a hypertrophic scar tissue as described in claim 9, wherein said step of injecting said hypertrophic scar tissue underlying a plurality of injection locations between about zero centimeters and about two centimeters distance apart on the skin of an animal with an amount of said efficacious concentration of said plurality of viable fibroblast cells in said amount of diluent comprises the step of injecting said hypertrophic scar tissue underlying a plurality of injection locations between about zero centimeters and about two centimeters distance apart on the skin of an animal with an amount of said efficacious concentration of said plurality of viable fibroblast cells in said amount of diluent of about 0.05 milliliters and about 0.2 milliliters.

11. The method for treatment of a hypertrophic scar tissue as described in claim 10, further comprising the step of establishing said amount of said efficacious concentration of said plurality of viable fibroblast cells in said amount of diluent of about 0.05 milliliters and about 0.2 milliliters along an injection needle withdrawal path in said hypertrophic restrictive scar tissue.

12. The method for treatment of a hypertrophic scar tissue as described in claim 10, wherein said step of injecting said hypertrophic scar tissue underlying a plurality of injection locations between about zero centimeter and about two centimeters distance apart on the skin of an animal with an amount of said efficacious concentration of said plurality of viable fibroblast cells in said amount of diluent of about 0.05 milliliters and about 0.2 milliliters comprises the step of injecting said hypertrophic scar tissue underlying a plurality of injection locations between about zero centimeter and about two centimeters distance apart on the skin of an animal with an amount of said efficacious concentration of said plurality of viable fibroblast cells in said amount of diluent of about 0.05 milliliters and about 0.2 milliliters which includes a concentration of said plurality of viable fibroblast cells of between about five million fibroblast cells per milliliter and about twenty million fibroblast cells per milliliter of diluent.

13. A hypertrophic scar tissue treatment comprising an efficacious concentration of a plurality of viable fibroblast cells in an amount of diluent injectable into an amount of hypertrophic scar tissue underlying an injection location on a skin surface.

14. The hypertrophic scar tissue treatment as described in claim 13, wherein said efficacious concentration of said plurality of viable fibroblast cells in said amount of diluent comprises a concentration of said plurality of viable autologous fibroblast cells in said amount of diluent of not less than about one million viable fibroblast cells per milliliter of said diluent.

15. The hypertrophic scar tissue treatment as described in claim 13, wherein said efficacious concentration of said plurality of viable fibroblast cells in said amount of diluent comprises a concentration of said plurality of viable autologous fibroblast cells in said amount of diluent of between about five million and about twenty million viable autologous fibroblast cells per milliliter of said diluent.

16. The hypertrophic scar tissue treatment as described in claim 14, wherein said an amount of diluent containing said efficacious concentration of said plurality of viable autologous fibroblast cells injectable into an amount of hypertrophic scar tissue underlying said injection location on said skin surface comprises an amount of diluent of between about 0.05 milliliters and about 0.2 milliliters.

17. The hypertrophic scar tissue treatment as described in any one of claim 13, 14, 15, or 16, wherein said an efficacious concentration of a plurality of viable fibroblast cells in an amount of diluent injectable into an amount of hypertrophic scar tissue underlying an injection location on a skin surface comprises an efficacious concentration of a plurality of viable autologous fibroblast cells in an amount of diluent injectable into an amount of hypertrophic scar tissue underlying an injection location on a skin surface.

18. The hypertrophic scar tissue treatment as described in any one of claim 13, 14, 15, or 16, wherein said an efficacious concentration of a plurality of viable fibroblast cells in an amount of diluent injectable into an amount of hypertrophic scar tissue underlying an injection location on a skin surface comprises an efficacious concentration of a plurality of viable allogenic fibroblast cells in an amount of diluent injectable into an amount of hypertrophic scar tissue underlying an injection location on a skin surface.
